(19) United States
(12) Patent Application Publication
(10) Pub. No.: US 2018/0078461 A1
(43) Pub. Date: Mar. 22, 2018

(54) JEJUNOSTOMY TUBE AND METHOD

(71) Applicant: Health Research, Inc., Buffalo, NY (US)

(72) Inventors: Steven HOCHWALD, Buffalo, NY (US); Smit SINGLA, Buffalo, NY (US); Sergei KURENOV, Buffalo, NY (US)

(21) Appl. No.: 15/564,103

(22) PCT Filed: Apr. 4, 2016
(86) PCT No.: PCT/US16/25886

§ 371 Date: Oct. 3, 2017

Related U.S. Application Data
(60) Provisional application No. 62/143,048, filed on Apr. 4, 2015.

(51) Int. Cl. A61J 15/00

(52) U.S. Cl. CPC A61J 15/0015; A61J 15/0069; A61B 17/00234; A61J 15/0092; A61J 15/0038

(57) ABSTRACT

A jejunostomy tube (“J-tube”) is presented. The J-tube is amenable for placement via total laparoscopic approach, without the need for any percutaneous, endoscopic, or open operative assistance. The presently-disclosed device allows delivery of nutrients, water, and medications to the intestinal tract of an individual in a safe manner, without the disadvantages associated with the use of conventional tubes.

### CROSS-REFERENCE TO RELATED APPLICATIONS

This application claims priority to U.S. Provisional Application No. 62/143,048, filed on Apr. 4, 2015, now pending, the disclosure of which is incorporated herein by reference.

### FIELD OF THE DISCLOSURE

The present disclosure relates to feeding tubes, and more particularly to jejunostomy tubes.

### BACKGROUND OF THE DISCLOSURE

In the last few years, there has been an increased demand for performing minimally-invasive surgeries using, for example, the laparoscope. The number of operations performed laparoscopically has risen steadily, and with the introduction of advanced laparoscopic and robotic training in residency and fellowships, this number will continue to rise further.

During times of illness secondary to conditions like cancer, trauma, stroke or neurological dysfunction, many patients are unable to orally ingest food, water, medications, or other ingestible materials that aid in recovery and treatment. In such situations, feeding tubes are placed to allow for delivery of such ingestible materials. Multiple research studies have shown that a feeding tube can successfully provide a safe medium for delivery of nutrition or medications during acute illness, which hastens recovery and allows early healing. Once the patient recovers, the feeding tube is removed easily in the office without the need for another operation.

For short-term use, feeding tubes are placed via the mouth or more commonly, via the nose. However, for long-term use, these routes are not patient friendly, and therefore long-term feeding tubes are placed into either the stomach or the jejunum. Feeding tubes placed in the intestinal tract are generally placed via an open operation, which increases recovery time requiring hospital stays of 1-2 days. The risk of complication is also increased.

A minimally-invasive laparoscopic approach utilizing small incisions has been used to reduce the risk of complications and allow for same-day discharge from the hospital. At present, other tubes, such as biliary drain tubes (“T-tubes”), are modified to allow for placement via the laparoscopic approach. However, there are some inherent issues that are frequently seen with the use of these conventional tubes. First, such tubes have to be modified each time before use as a J-tube, causing variation in size, shape, and other geometry. Second, due to their function for drainage, tubes such as T-tubes are not adapted for connection to other equipment. When connections are made, they are weak and can become loose. Additionally, between uses, such tubes are capped, and such caps are frequently dislodged causing inconvenience to patients due persistent reflux and leakage.

### BRIEF SUMMARY OF THE DISCLOSURE

A jejunostomy tube (“J-tube”) is presented, the J-tube is amenable for placement via total laparoscopic approach, without the need for any percutaneous, endoscopic or open operative assistance. This device allows delivery of nutrients, water, and medications to the intestinal tract in a safe manner, without the disadvantages associated with the use of conventional tubes.

### DESCRIPTION OF THE DRAWINGS

For a fuller understanding of the nature and objects of the disclosure, reference should be made to the following detailed description taken in conjunction with the accompanying drawings, in which:

FIG. 1 depicts a jejunostomy tube (“J-tube”) according to an embodiment of the present disclosure;

FIG. 2 is a diagram of a portion of a J-tube showing the proximal end;

FIG. 3 is a diagram of a portion of a J-tube showing the distal end;

FIG. 4A is a end-view diagram of the distal end of FIG. 3;

FIG. 4B is an top-view diagram of the distal end of FIG. 4A;

FIG. 5 is a diagram of a portion of a J-tube having a stylet according to another embodiment of the present disclosure;

FIG. 6 is another view of the J-Tube of FIG. 5; and

FIG. 7 is a chart of a method according to another aspect of the present disclosure.

### DETAILED DESCRIPTION OF THE DISCLOSURE

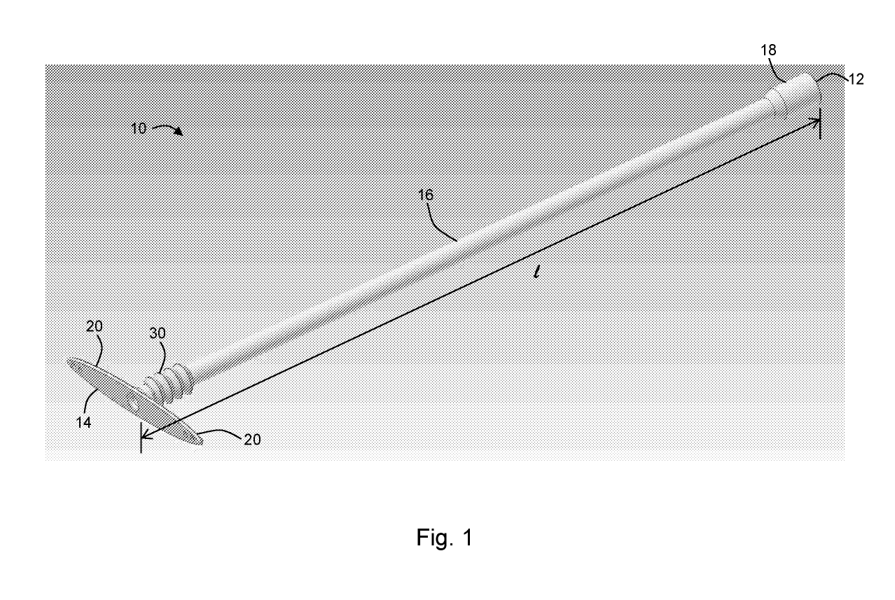

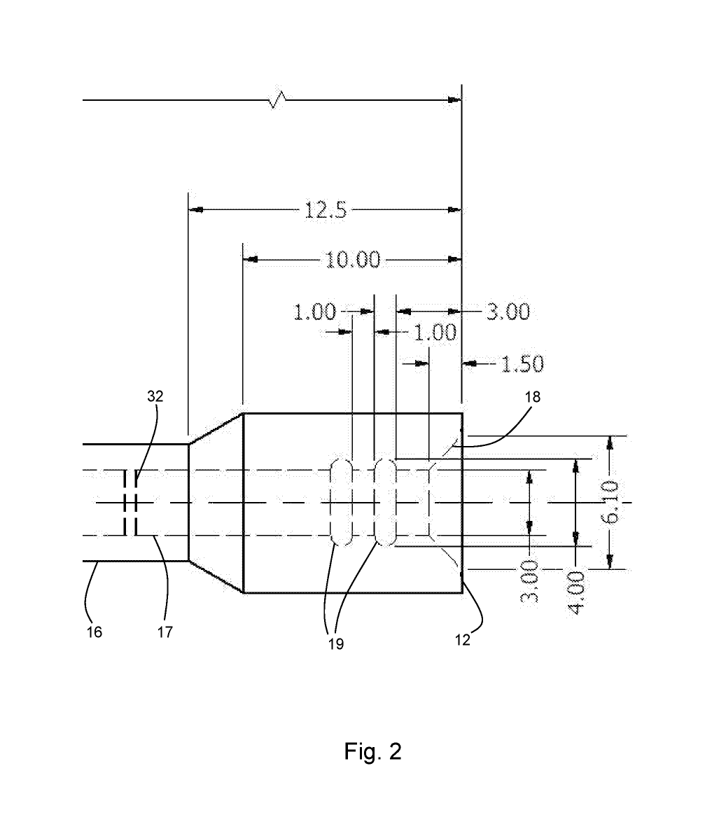

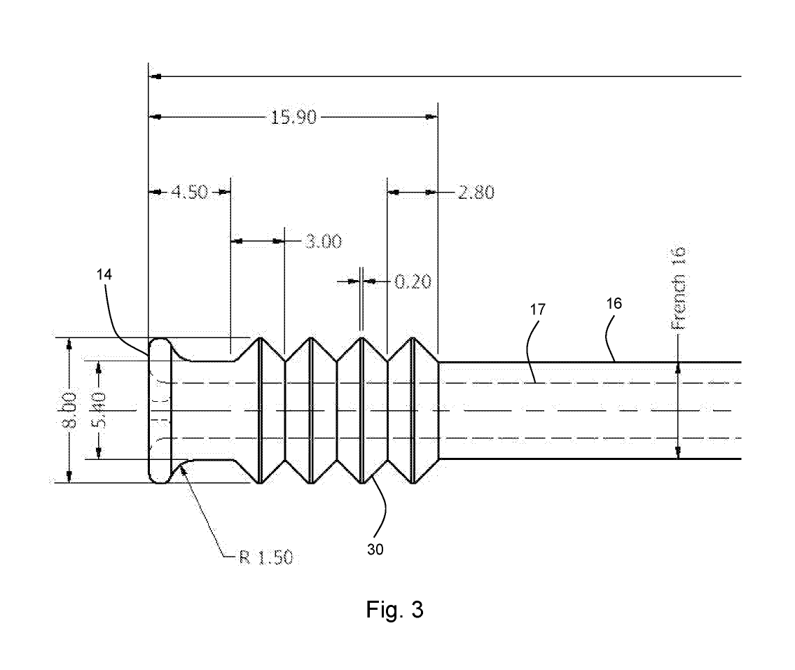

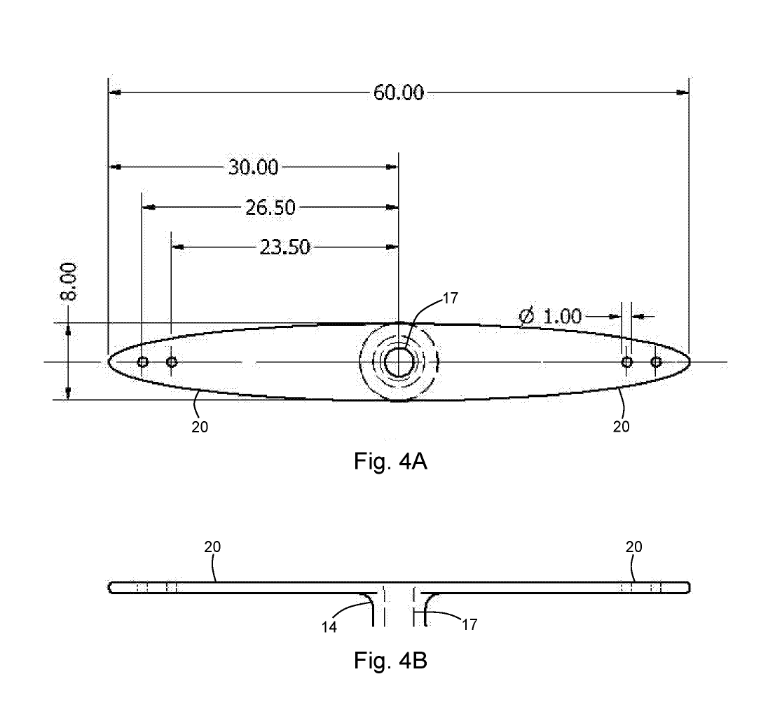

With reference to FIG. 1, the present disclosure may be embodied as a laparoscopy-compatible jejunostomy tube (“J-tube”) 10. Such an intestinal feeding tube 10 is capable of placement in an individual by way of a total laparoscopic approach—i.e., capable of placement into the peritoneal cavity using standard laparoscopic or robotic ports. The jejunostomy tube 10 has a body 16 having a proximal end 12 to a distal end 14. The body 16 includes a fluid passage 17 (see, e.g., FIG. 2) extending therethrough. The body 16 has a length (l) which may be any length suitable to a particular use as will be apparent to one having skill in the art in light of the present disclosure. In some embodiments, the length of the body 16 may be between 15 cm and 25 cm. In some embodiments of the J-tube 10, the body 16 has a length of 18.5 cm. The body 16 may be tubular. The J-tube 10 may be of any diameter according to the intended use. In some embodiments, the J-tube 10 is 15 French or 16 French (15fr or 16fr). The J-tube 10 may be narrower or wider as appropriate. Components of the J-tube 10 may be made from polyurethane, silicone, latex-free rubber, or other materials, including flexible materials, and combinations thereof.

The proximal end 12 serves as an input and lies on the outside of the individual's body. Ingestible materials can be delivered through the J-tube 10 by connecting an inlet 18 (described below) with a syringe, a tube attached to a pump, or other devices suitable for providing ingestible materials.

The distal end 14 of the body 16 may include at least one appendage 20 attached to the body 16. In some embodiments, the J-tube 10 comprises two appendages 20 attached to the body 16 at the distal end 14. The appendages 20 are configured to fold, for example, against the body 16, together beyond the distal end 14, or otherwise, while the J-tube 10 placed through an incision. The appendages 20 can be biased, such as, for example, by a resilience of the appendage material, to extend to a position generally orthogonal to the body 16 once placed into the jejunum. In this way, the J-tube 10 is generally prevented from unintentional removal due to the action of the appendage(s) 20 against the inner wall of the intestine, where the appendages 20 act as retainers to maintain the distal end 14 of the J-tube 10 in the intestinal lumen.

The appendages 20 are further configured to fold into a removal configuration (e.g., folded together at a position beyond the distal end 14, substantially parallel to the body 16, etc.) when a removal force is applied to the J-tube 10, such as, for example, a pulling force applied by a medical professional for removing the J-tube 10 from the individual. In some embodiments, the appendages 20 have rolled edges. Such rolled edges may, among other things, enhance the compatibility of the appendages 20 with the intestinal wall and/or aid in the biasing force (causing extension on deployment) of the appendages 20.

In an exemplary embodiment, a J-tube 10 has two generally triangular appendages 20, each appendage 20 having a length of 30 mm. The width of each appendage 20 is 7 mm where attached to the body 16, tapering to a width of 2 mm at a rounded end further away from the body 16. The appendages 20 are attached to the body 16 at locations on the distal end 14 which are radially opposite from one another.

The proximal end 12 of the J-tube 10 includes an inlet 18 configured to connect to other equipment. For example, the inlet 18 may be used to connect the J-tube 10 to a syringe, a pump, or other components used to deliver ingestible material to the individual. The inlet 18 may comprise one or more grooves 19 on an internal surface of the inlet 18, the grooves 19 being configured to provide an enhanced connection with connected equipment. Such a configuration reduces the occurrence of slippage seen with conventional feeding tubes. In some embodiments, a plurality of grooves 19 may be conically shaped to provide a tapered interaction when connected to a device.

The J-tube 10 may comprise flaps 30 on an external surface of the body 16 and configured to prevent leakage/reflux of intestinal contents from around a perimeter of the body 16 where the body 16 enters the intestine. As such, the flaps 30 are at a location on the body 16 which is spaced apart from the distal end 14.

The J-tube 10 may further comprise a valve 32, such as a one-way valve. The valve 32 is disposed in the fluid passage 17 of the body 16 and is configured to allow fluid flow from the proximal end 12 to the distal end 14, but prevent a reverse flow from the distal end 14 to the proximal end 12. In this way, ingestible material may be provided to the individual and leakage through the J-tube 10 may be avoided. The valve 32 may be, for example, a membrane valve located near the inlet 18 which is penetrable (for actuation of the valve 32) by a device inserted into the inlet 18, and prevents egress of fluid once the device is removed.

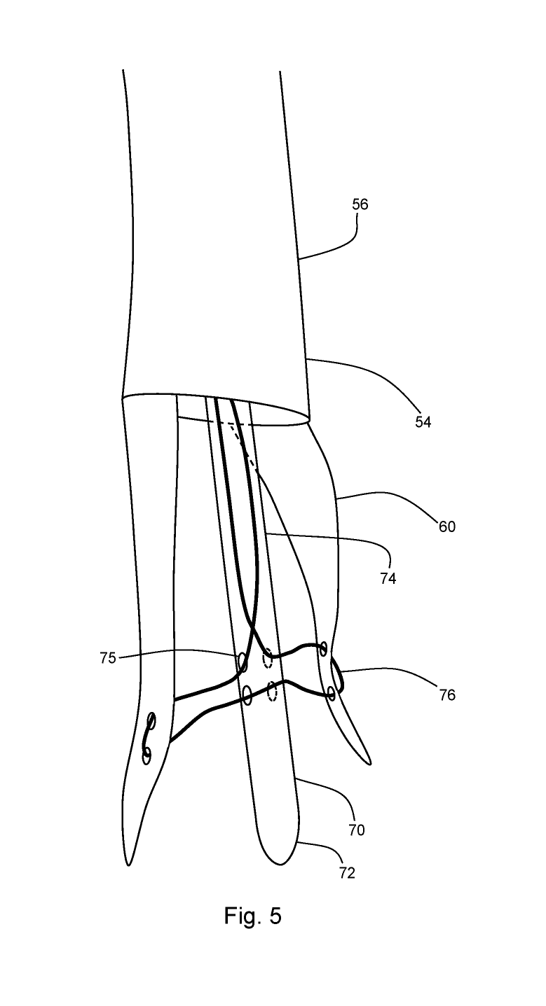

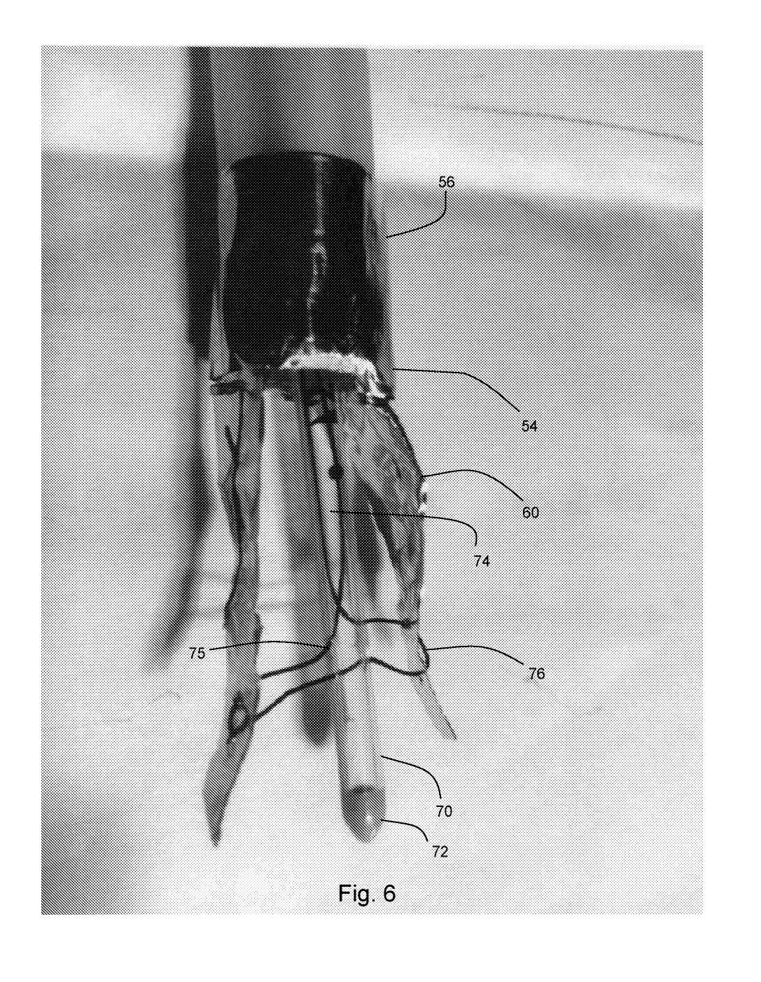

Embodiments of a presently disclosed J-tube may further comprise a stylet 70 to facilitate placement of the J-tube (see, e.g., FIGS. 5 and 6). In such embodiments, the stylet 70 runs through the fluid passage of the body 56. The stylet 70 has an operable end and a head end 72 which may be conically shaped. The stylet 70 further comprises a passage 74 and at least one orifice 75 at an end of the passage 74 proximal to the head end 72. A string 76 is disposed through the passage 74 and orifice 75 and attached to a tip of the appendage 60. In some configurations, the string is looped through the tip of the appendage 60 such that both ends of the string 76 exit the stylet 70 at the operable end. The string 76 is operable to hold the appendage 60 into a folded position during placement of the J-tube.

Once the distal end 54 of the J-tube is positioned within the intestinal lumen, the string 76 is cut to allow the appendage 60 to extend as described above, and the string 76 is withdrawn through the passage 74 and/or the stylet 70 is withdrawn from the body 56. The string 76 may be a non-absorbable nylon, or other materials known for such uses. The stylet 70 may comprise a number of orifices 75 and strings 76, corresponding to the number of appendages 60. In some embodiments, one string 76 is looped through each appendage 60 and disposed through one orifice 75. In this way, a single string 76 may be used to deploy multiple appendages 60 into their extended positions.

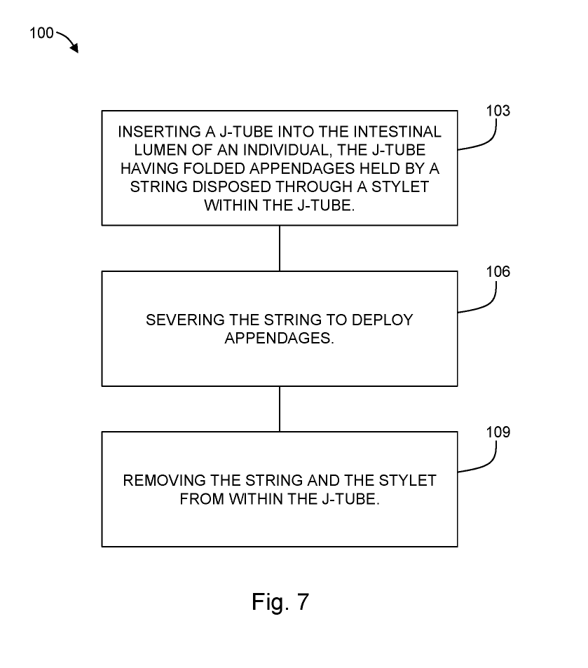

In another aspect (an example of which is depicted in FIG. 7), the present disclosure is a method 100 of laparoscopic deployment of a J-tube. The J-tube has extendable appendages at a distal end of the J-tube, and the appendages are maintained in a folded position by way of a string disposed through a stylet. The distal end of the J-tube is inserted 103 into the intestinal lumen of an individual, using the stylet. The distal end is inserted 103 through an incision. The method 100 includes severing 106 the string to allow the appendages to extend into a position substantially orthogonal to the J-tube. The stylet and string are removed 109 from the J-Tube.

Although the present disclosure has been described with respect to one or more particular embodiments, it will be understood that other embodiments of the present disclosure may be made without departing from the spirit and scope of the present disclosure. All such particular embodiments are intended to be non-limiting examples. Dimensions shown in the figures are exemplary and, as such, are not intended to be limiting.

What is claimed is:

1. A jejunostomy tube (J-tube) for laparoscopic placement in an individual, comprising:
a tubular body having a distal end and a proximal end;
one or more appendages attached to the tubular body at the distal end and configured to move from a folded position for deployment to an extended position generally orthogonal to the tubular body;
an inlet at the proximal end and in fluid communication with a fluid passage of the tubular body, the inlet configured for connection to a feeding device; and
a plurality of flaps disposed on an exterior surface of the tubular body, proximal to the one or more appendages, for preventing reflux along the exterior of the body.

2. The J-tube of claim 1, wherein the one or more appendages comprises two appendages.

3. The J-tube of claim 1, further comprising a valve disposed in the fluid passage of the tubular body and configured to permit fluid flow through the fluid passage from the proximal end to the distal end, and to prevent fluid flow through the fluid passage from the distal end to the proximal end.

4. The J-tube of claim 3, wherein the valve is a membrane valve configured to be penetrated by feeding equipment when connected at the inlet.

5. The J-tube of claim 1, wherein the one or more appendages have rolled edges.

6. The J-tube of claim 1, further comprising a stylet for deployment.

7. The J-tube of claim 6, wherein the stylet further comprises a string used to maintain the one or more appendages in a folded position during deployment and severable to cause extension of the one or more appendages when deployed.

8. The J-tube of claim 1, wherein the inlet comprises a plurality of grooves on an inner surface.

9. The J-tube of claim 8, wherein the plurality of grooves are conically shaped.

10. A method of laparoscopic deployment of a jejunostomy tube (“J-tube”) having extendable appendages at a distal end of the J-tube, the appendages maintained in a folded position by way of a string disposed through a stylet, the method comprising:
inserting the distal end of the J-tube into the intestinal lumen of an individual, using the stylet, wherein the distal end is inserted through an incision;
severing the string to allow the appendages to extend into a position substantially orthogonal to the J-tube; and
removing the stylet and string from the J-tube.
